(19) United States
(12) Patent Application Publication
(10) Pub. No.: US 2026/0263725 A1
(43) Pub. Date: Sep. 10, 2026

(54) METHOD AND EQUIPMENT FOR GENERATING NITRIC OXIDE GAS

(71) Applicant: Liwei Huang, Hangzhou (CN)

(72) Inventor: Liwei Huang, Hangzhou (CN)

(21) Appl. No.: 18/918,242

(22) PCT Filed: May 18, 2023
(86) PCT No.: PCT/CN2023/094949

§ 371 Date: Oct. 17, 2024

(30) Foreign Application Priority Data
May 20, 2022 (CN) 202210639823.3

(51) Int. Cl. A61M 16/10

(52) U.S. Cl. CPC A61M 16/10; A61M 2202/0275

(57) ABSTRACT

A method and equipment is disclosed for generating nitric oxide gas, which is related to the field of gas generation and medical treatment. The method is to mix a liquid medicament with a solid medicament to generate nitric oxide gas by chemical reaction, wherein the active ingredient of the solid medicament is ferric nitrosyl chloride or its hydrates, and the active ingredient of the liquid medicament is one of or a mixture of water, an alkali solution or slurry, a salt solution or slurry. Also disclosed is a corresponding equipment of the method for nitric oxide gas generation. The carbon dioxide gas generated during the chemical reaction not only can have the effect of diluting the concentration of nitric oxide gas, but also can have an auxiliary effect of enhancing respiration. The equipment is simple to operate, convenient to carry, and safe and reliable.

### TECHNICAL FIELD

The invention relates to a method and equipment for generating nitric oxide gas, in particular to the application in fields of nitric oxide gas generation and medical treatments.

### BACKGROUND

Nitric oxide (NO) is a colorless gas, which is slightly soluble in water. Previous studies have found that nitric oxide's fat-soluble properties allow it to function as a messenger molecule in human body. Nitric oxide molecule can easily penetrate through cell membranes, causing smooth muscle cells around blood vessels to receive signals and relax, resulting in the dilatation of blood vessels. Therefore, inhaling NO can be used as an assistant treatment method for heart and lung diseases, and is also regarded as a beneficial way for health-care. However, when the amount of inhalation of NO exceeds a certain concentration and quantity, it may be harmful to human body.

At present, there are three routes for nitric oxide production in industry, including direct synthesis, ammonia catalytic oxidation, and reaction of sodium nitrite and dilute sulfuric acid according to different uses, and the obtained nitric oxide gas is usually stored by pressurized status. When nitric oxide gas is used for medical treatment in hospital, it is usually stored in a gas cylinder and needs to be diluted in advance. Therefore, NO-inhaling treatment is generally suitable for centralized use in medical institutions, and is inconvenient for individual use at home. On the other hand, since the nitric oxide gas is active in chemical property and easy to convert into nitrogen dioxide and nitrous oxide, it is not suitable for long-term storage. In order to reduce the using cost and facilitate the use for individuals and families, many efforts have been made to develop portable nitric oxide gas generators. According to working principle, these portable nitric oxide gas generators are mainly classified as two types: one is by gas discharge technologies, such as China patent publication No. CN113456966A, and the other is by electrolysis of nitrite solutions, such as China patent publication No. CN111636071A. However, the operation requirements and high cost for these nitric oxide gas generators prevent its popularization into use.

The inventor of the present invention discloses a method for obtaining a new chemical compound by means of a chemical reaction between ferric chloride and nitric oxide gas (China patent No. ZL 201610541447.9 and No. ZL 201910889947.5), wherein the reaction product is considered to be ferric nitrosyl chloride or its hydrates. The present invention develops a nitric oxide gas generation method and equipment by using the compounds obtained in above inventions.

### SUMMARY OF THE INVENTION

In view of the problems existing in the prior art, the objective of the present invention is to provide a technical solution for providing a method and equipment for generating nitric oxide gas.

The present invention adopts the following technical solution: A method for generating nitric oxide gas is characterized in that a liquid medicament is mixed with a solid medicament to generate nitric oxide gas by chemical reaction. The active ingredient of said solid medicament is ferric nitrosyl chloride (Fe(NO)ₓCl₃, x≤3) or its hydrates, which may be the product of the reaction between ferric chloride and nitric oxide. A certain amount of auxiliary components may also be added to the solid medicament, which are generally acidic salts (mainly transition metal sulfates and chlorides), including ferric (or ferrous) chloride, aluminum chloride, ferric (or ferrous) sulfate, and aluminum sulfate. The amount of the addition of the auxiliary component is determined according to needs. One of the functions of the auxiliary component is to dilute the content of the active ingredient, and the other is to react with the liquid medicament to generate a gas to dilute the produced nitric oxide gas. The liquid medicament includes water, alkali solutions or slurries, salt solutions or slurries, or the mixture of above compounds. Said salt comprises carbonates of alkali metals, alkaline earth metals and a transition metals, and corresponding acid salts, basic salts or complex salts, preferably alkali metal carbonates, such as sodium carbonate and potassium carbonate.

When the alkaline solutions or slurries are used as the liquid medicament, the reaction between solid medicament and liquid medicament may be enhanced with high pH value of the liquid medicament. Preferably, the pH value of the alkaline solution or slurries is 10-12. When carbonate solutions or slurries are used as the liquid medicament, carbon dioxide gas will be generated by the reactions between the carbonates and the active ingredient or/and the auxiliary ingredient of the solid medicament, which may also play a role in diluting the produced nitric oxide gas, so that the oxidation of the nitric oxide to harmful nitrogen dioxide by oxygen in air may be prevented. When the active ingredient of ferric nitrosyl chloride (Fe(NO)Cl₃) and the auxiliary component of ferric chloride are used as solid medicament, and sodium carbonate solution is used as liquid medicament, the main reactions are:

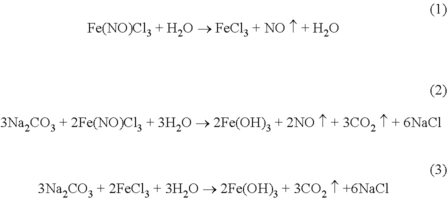

Wherein, reaction equation (1) is a hydrolysis reaction of ferric nitrosyl chloride, and reaction equation (2) and reaction formula (3) are neutralization reaction. In order to control the release amount and concentration of NO, the amounts of active ingredient and the auxiliary ingredient in the solid medicament can be proportioned as required. For medical use, the content of the active ingredient of ferric nitrosyl chloride in the solid medicament is generally 10% or less, but is not limited. The water content in solid medicament is generally less than about 20%. The storage temperature of the solid medicament is commonly below 50° C. (maybe melted at high temperature), and the storage temperature of the liquid medicament is above its freezing point, typically above 0° C. The containers of the medicaments may be filling by inert gases such as nitrogen.

When the produced gas is used for medical treatment, after diluted by air, the concentration of NO may be less than or equal to 100 ppm, and the concentration of CO₂ is less than or equal to 2%. Preferably, CO₂ concentration is in the range of 0.1-1%. The concentration of nitric oxide and flow rate of the generated gas may also be adjusted by controlling the content of carbonate in liquid medicament and the flow rate of the liquid medicament during the solid-liquid reaction.

The liquid medicament can also be prepared by maxing the liquid which does not chemically react with the solid medicament, such as glycerol to slow down the solid-liquid reaction.

An equipment of said method to generate nitric oxide gas comprises a solid medicament container, an outer container, a liquid medicament container and connecting tubes (pipes) and valves (regulators). The solid medicament container is fixed at the bottom of the outer container. The liquid medicament container is arranged at the upper part of the equipment, and connecting with outer container via a connecting tube, wherein a liquid medicament flow regulator is installed on the connecting tube. There is a produced gas release tube with a gas release valve connecting the outer container at the upper side of the outer container, and a gas filter is further arranged at the upper part in the outer container, which is used for removing mist possibly generated from liquid-solid reaction process.

The solid medicament container is a U-shaped cylinder, the upper end of which is open and is used for receiving the liquid medicament from the connecting tube. There is an empty space (about ⅕~ 1/10 of the volume) left at the upper part of the solid medicament container to receive the liquid medicament. The liquid after the reaction may be discharged from the upper end of the solid medicament container, or from the holes on the wall of the solid medicament container. The numbers and positions of the holes may be arranged depending on requirement. The holes on the wall of the container may be vertically arranged in a staggered distribution. Or, a shielding ring may be arranged above the holes so as to be free from the influence of the liquid flowing down from upper holes. The annulus space between the solid medicament container and the outer container is used to receive the reaction products, and the volume may receive all of the produced liquid. The outer container may be a little bit bigger to be effected as a buffer to dilute the produced gases.

Using said equipment to generate the gas according to said method follows next steps: firstly open the gas release valve on the release tube of the outer container, and then open the vent valve, the liquid outlet valve and the liquid inlet valve, in sequence, so that the liquid medicament from the liquid medicament container enters into the solid medicament container through the connecting pipe and then reacts with the solid medicament to generate a mixed gas of nitric oxide and carbon dioxide, and then the produced gas is released by the gas release tube via said filter. When the equipment is used as a first-aid device, the generated gas can be naturally inhaled together with the surrounding air. Based on the inhalation air volume of 5 to 10 L/min for a normal adult, the dilution times after inhalation can usually be set to 50 to 200. The concentration of NO may be less than 100 ppm, and the concentration of CO₂ is generally less than or equal to 2% after dilution. The control of the concentration of nitric oxide and flow rate of the generated gas may be achieved by adjusting the flow rate of the liquid medicament.

The empty space of the containers may be filled with inert gas such as nitrogen gas, which may be effect as protection and dilution gas. The concentration of produced nitric oxide gas can be controlled by adjusting the flow rates of the liquid medicament by the flow regulator, which may be selective as large, medium, and small flow rate, depending on the requirements. The release of nitric oxide gas can also be controlled by changing the solid-liquid contact area, such as changing the cross-sectional area of the solid medicament container or adding an amount of a neutral substance such as quartz sand to the solid reactant. The solid-liquid reaction rate may be reduced by half, when the solid-liquid contact area is reduced by half.

The merit of present invention is that the solid medicament and the liquid medicament are encapsulated in different containers, and carbon dioxide gas generated during the reaction between the liquid-solid reaction plays a role of diluting the concentration of nitric oxide gas to avoid to be oxidized to toxic nitrogen dioxide gas. The produced carbon dioxide gas can also play a role in enhancing respiration.

### BRIEF DESCRIPTION OF THE DRAWINGS

FIG. 1 is a schematic structural diagram of the equipment for generating a nitric oxide gas.

FIG. 1 illustration: 1 solid medicament; 2 solid medicament container; 3 outer container; 4 connecting tube; 5 filter; 6 liquid inlet valve; 7 liquid medicament flow regulator; 8 liquid outlet valve; 9 liquid medicament; 10 liquid medicament container; 11 vent valve; 12 gas release valve; 13 gas release tube.

### DETAILED DESCRIPTION OF THE PREFERRED EMBODIMENTS

The present invention will be described in further detail below with reference to the accompanying drawings and embodiments.

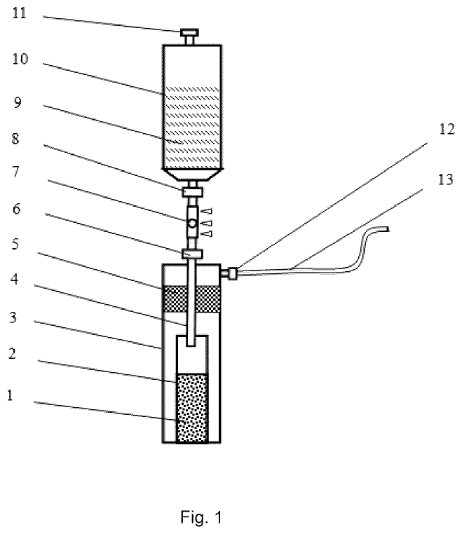

An equipment for generating a nitric oxide gas is shown in FIG. 1, which includes solid medicament 1, a solid medicament container 2, an outer container 3, a connecting tube 4, a filter 5, a liquid inlet valve 6, a liquid medicament flow regulator 7, a liquid outlet valve 8, a liquid medicament 9, a liquid medicament container 10, a vent valve 11, a gas release valve 12 and a gas release tube 13.

The solid medicament container 2 is placed in the outer container 3, and both as the lower part of the generator. The liquid medicament container 10 is as the upper part of the generator with a vent valve 11 arranged at the top of the container, and a liquid medicament outlet 8 at the bottom part of the container. The liquid medicament outlet is communicated with the outer container by a connecting tube 4 and a liquid inlet valve 6. A liquid medicament flow regulator 7 is arranged on the connecting tube for the controlling of the flow rate of liquid medicament. A gas release tube 13 with a gas release valve 12 is connected the outer container at the upper side of the outer container, and a gas filter 5 is further arranged at the upper part in the outer container.

The solid medicament container 2 with solid medicament 1 is a U-shaped cylinder, the upper end of which is open and is used for receiving the liquid medicament from the connecting tube 4. There is an empty space left at the upper part of the solid medicament container to receive the liquid medicament discharged from liquid medicament container. The annulus space between the solid medicament container and the outer container is used to receive the reaction products.

The operation steps are as follows: firstly, open the gas release valve 12, and then open the vent valve 11, the liquid outlet valve 8 and the liquid inlet valve 6 in sequence, so that the liquid medicament enters into the solid medicament container 2 through the connecting tube 4 and the liquid medicament flow regulator 7 to react with the solid medicament to generate a mixed gas of nitric oxide and carbon dioxide. The produced gas is released by the gas release tube 13 through the gas release valve 12 and filter 5. The control of the concentration of the generated nitric oxide gas may be achieved by adjusting the liquid medicament flow regulator 7.

Example 1: An equipment of the method for the generation of nitric oxide gas is shown in FIG. 1. All the containers are cylindrical and made of polyethylene plastic (HDPE). The connecting tube and gas release tube are silicone tubes. The dimension of liquid medicament container is about 80 mm in diameter and 100 mm in height. The dimension of outer container is about 100 mm in diameter and 150 mm in height, the filter in outer container is made of absorbent cotton with a thickness of about 20 mm (containing about a height of 5 mm alumina particle layer in the filter to remove acidic gas). The dimension of the solid medicament container is about 30 mm in diameter and 90 mm in height. The filling height of the solid medicament in the container is about 60 mm (after being filled with slurry, condensed and solidified). The content of the effective component of nitrosyl ferric chloride (Fe(NO)Cl₃) in the solid medicament is about 0.6%, with moisture about 10%, and ferric chloride the balance. A volume of 400 mL sodium carbonate solution with a mass concentration of 22% is used as the liquid medicament. The diameter of the gas release tube is 8 mm. The containers are filled with atmospheric nitrogen for protection. The operation is at room temperature of about 25° C. The flow rate of the liquid medicament is about 8 ml/min. The average gas flow rate of the produced gas discharging from the gas release tube is about 100 mL/min, and the average concentration of nitric oxide is 1900±500 ppm. The concentration of nitric oxide is decrease to about 19±5 ppm after dilution 100 times by air, and the CO₂ concentration is less than or equal to 1%. The total release time is about 50 min.

Example 2: The contents of the compositions in the solid medicament are effective component nitrosyl ferric chloride about 0.3%, quartz sand 20%, moisture about 10%, iron sulfate about 20%, and the balance ferric chloride. Except the liquid flow rate is about 4 mL/min, other conditions are the same as in example 1. The average gas flow rate of the produced gas discharging from the gas release tube is about 50 mL/min, and the average concentration of nitric oxide is 1200±300 ppm. The concentration of nitric oxide is decrease to about 6±3 ppm after dilution 200 times by air, and the CO₂ concentration is less than or equal to 0.5%. The total release time is about 100 min.

The invention claimed is:

1. A method for generating nitric oxide gas, wherein a liquid medicament is mixed with a solid medicament to generate a gas mixture of nitric oxide and carbon dioxide by chemical reaction, wherein the active ingredient of the solid medicament is ferric nitrosyl chloride (Fe(NO)xCl3, x≤3) or its hydrates, and the active ingredient of the liquid medicament is the carbonate of an alkali metal, an alkaline earth metals and a transition metals, or its corresponding acid salt, basic salt and complex salt, or a mixture of above said compounds.

2. (canceled)

3. The method for generating nitric oxide gas according to claim 1, wherein the solid medicament contains acidic salts as an auxiliary component, including sulfates and chlorides of the transition metals.

4. The method for generating nitric oxide gas according to claim 1, wherein the solid medicament contains at least one of the compounds of ferric chloride, ferrous chloride, aluminum chloride, ferric sulfate, ferrous sulfate and aluminum sulfate.

5. (canceled)

6. The method for generating nitric oxide gas according to claim 1, wherein the carbonate of the alkali metal is sodium carbonate or potassium carbonate.

7. (canceled)

8. (canceled)

9. (canceled)

10. The method for generating nitrogen monoxide gas according to claim 1, wherein a equipment of said method comprises solid medicament container (2), outer container (3), connecting tube (4), filter (5), liquid inlet valve (6), liquid medicament flow regulator (7), liquid medicament outlet valve (8), liquid medicament (9), liquid medicament container (10), vent valve (11), gas release valve (12) and gas release tube (13), wherein the cylinder-shaped solid medicament container (2) is fixed at the bottom of the outer container, with the solid medicament (1) loaded in the solid medicament container directly facing the nozzle of the connecting tube, and the liquid medicament container (10) is connected to the outer container through the connecting pipe (4);
wherein the method using the equipment follows next steps: firstly, open the gas release valve (12) of the outer container (3), and then open the vent valve (11), the liquid outlet valve (8) and the liquid inlet valve (6) in sequence, so that the liquid medicament (9) enters the solid medicament container (2) through the connecting tube (4) and reacts with the solid medicament to generate a gas mixture of nitric oxide and carbon dioxide, and then the produced gas is guided out from the gas release valve (12) and the gas release tube (13).
